(19) United States
(12) Patent Application Publication
(10) Pub. No.: US 2005/0004503 A1
(43) Pub. Date: Jan. 6, 2005

(54) Method and apparatus for treating acute myocardial infarction with hypothermic perfusion

(76) Inventors: Wilfred J. Samson, Saratoga, CA (US); Janine Robinson, Half Moon Bay, CA (US); Brady Esch, San Jose, CA (US)

Correspondence Address: FULWIDER PATTON LEE & UTECHT, LLP, 200 OCEANGATE, SUITE 1550, LONG BEACH, CA, 90802, US

(21) Appl. No.: 10/897,491

(22) Filed: Jul. 23, 2004

Related U.S. Application Data
(63) Continuation-in-part of application No. 10/102,124, filed on Mar. 19, 2002, now abandoned.
Application No. 10/102,124 is a continuation-in-part of application No. 09/384,467, filed on Aug. 27, 1999, now Pat. No. 6,673,040.
(63) Continuation-in-part of application No. 09/368,450, filed on Aug. 4, 1999, now Pat. No. 6,726,651.
(60) Provisional application No. 60/098,724, filed on Sep. 1, 1998.

(51) Int. Cl. A61M 37/00

(52) U.S. Cl. 604/6.14

(57) ABSTRACT

An apparatus and method are described for quickly inducing therapeutic hypothermia of the heart by perfusing the myocardium with hypothermic fluid in alternatingly antegrade and retrograde directions. The apparatus and method provide rapid cooling of the affected myocardium to achieve optimal myocardial salvage in a patient experiencing acute myocardial infarction. The therapeutic hypothermia system includes one or more coronary artery perfusion catheters, a coronary sinus perfusion catheter and a fluid source for delivering a hypothermically-cooled physiologically-acceptable fluid, such as saline solution, oxygenated venous blood, autologously-oxygenated arterial blood and/or an oxygenated blood substitute. The system may also include one or more guidewires, subselective catheters and/or interventional catheters introduced through a lumen in one or more of the perfusion catheters.

### CROSS-REFERENCE TO RELATED APPLICATION

This application is a continuation-in-part of U.S. patent application Ser. No. 10/102,124, filed Mar. 19, 2002 which is a continuation-in-part of U.S. patent application Ser. No. 09/384,467, filed on Aug. 27, 1999, which claims the benefit of U.S. provisional application Ser. No. 60/098,724, filed on Sep. 1, 1998, and a continuation-in-part of U.S. patent application Ser. No. 09/368,450 filed on Aug. 4, 1999, the specifications of which are hereby incorporated herein in their entirety.

### FIELD OF THE INVENTION

The present invention relates generally to methods and devices for treatment of heart disease. More particularly, it relates to methods and devices for treating acute myocardial infarction with hypothermic perfusion.

### BACKGROUND OF THE INVENTION

Heart disease is the most common cause of death in the United States and in most countries of the western world. Coronary artery disease accounts for a large proportion of the deaths due to heart disease. Coronary artery disease is a form of atherosclerosis in which lipids, cholesterol and other materials deposit in the arterial walls forming occlusions (blockages) that gradually narrow the arterial lumen, thereby depriving the myocardial tissue downstream from the normal blood flow that supplies oxygen and other critical nutrients and electrolytes. These conditions can be further exacerbated by an acute blockage due to thrombosis, principally caused by plaque rupture, which results in a severe reduction or blockage of blood flow that leads to ischemia. The cell damage that occurs due to ischemia is a biphasic process: initial ischemic damage followed by reperfusion injury. Reperfusion injury is paradoxical and the precise mechanism of it is not known, but the principle mediators appear to be cyctotoxic oxygen-derived free radicals and neutrophils; both initiate a cascade that results in stasis and microvascular plugging (no-reflow). The location of the occlusion and the length of time elapsed before treatment determines the tissue at risk and proportion of necrotic tissue.

Recent research has indicated that, during the acute stages of myocardial infarction, as much as half of the myocardial tissue at risk can be salvaged by hypothermic treatment. It is theorized that hypothermia retards the impact of reperfusion injury and may halt the progression of apoptosis, or programmed cell death. To date, most attempts at hypothermic treatment for acute myocardial infarction have involved total body hypothermia, for example using a blood heat exchanger inserted into the patient's vena cava. While this method has shown some efficacy in initial trials, it has a number of drawbacks. In particular, the need to cool the thermal mass of the patient's entire body slows the process, delaying the therapeutic effects of hypothermia. The more timely the patient's heart is cooled and timed with interventional reperfusion, the more myocardial tissue can be successfully salvaged.

Recent research has indicated that, during the acute stages of myocardial infarction, as much as half of the myocardial tissue at risk can be salvaged by hypothermic treatment of the ischemic area. It is theorized that hypothermia halts the progression of apoptosis or programmed cell death, which causes as much tissue necrosis as the ischemia that precipitated the myocardial infarction. To date, most attempts at hypothermic treatment for acute myocardial infarction have involved global hypothermia of the patient's entire body, for example using a blood heat exchanger inserted into the patient's vena cava. While this method has shown some efficacy in initial trials, it has a number of drawbacks. In particular, the need to cool the patient's entire body with the heat exchanger slows the process and delays the therapeutic effects of hypothermia. The more quickly the patient's heart can be cooled, the more myocardial tissue can be successfully salvaged.

Global hypothermia has another disadvantage in that it can trigger shivering in the patient. A number of strategies have been devised to stop the patient from shivering, but these add to the complexity of the procedure and have additional risk associated with them. Shivering can be avoided altogether by induction of localized hypothermia of the heart or of the affected myocardium without global hypothermia. Localized hypothermia has the additional advantage that it can be achieved quickly because of the lower thermal mass of the heart compared to the patient's entire body. Rapid induction of therapeutic hypothermia gives the best chance of achieving the most myocardial salvage and therefore a better chance of a satisfactory recovery of the patient after acute myocardial infarction.

What would be desirable, but heretofore unavailable, is an apparatus and method for rapid induction of therapeutic hypothermia of the heart or of the affected myocardium in a patient experiencing acute myocardial infarction.

### SUMMARY OF THE INVENTION

In keeping with the foregoing discussion, the present invention provides an apparatus and method for induction of therapeutic hypothermia of the heart by hypothermic perfusion of the myocardium, and more particularly, by subjecting the myocardium to alternatingly antegrade and retrograde flow of hypothermic fluid. The apparatus and method provide rapid cooling of the affected myocardium to achieve optimal myocardial salvage in a patient experiencing acute myocardial infarction.

The apparatus takes the form of a therapeutic hypothermia system including at least a coronary artery perfusion catheter, a coronary sinus perfusion catheter, a fluid source for delivering a hypothermically-cooled physiologically-acceptable fluid and a mechanism for alternatingly supplying the fluid through the two catheters. The coronary artery perfusion catheter has an elongated catheter shaft configured for transluminal introduction via an arterial insertion site, such as a femoral, subclavian or brachial artery. The coronary sinus perfusion catheter has an elongated catheter shaft configured for transluminal introduction via a venous insertion site such as the femoral or jugular vein. The proximal end of each catheter shaft has a perfusion fitting configured for connecting to the fluid source.

The distal end of the coronary artery perfusion catheter shaft is preferably curved to selectively engage either the right or the left coronary artery. A perfusion lumen extends through the catheter shaft from the perfusion fitting at the proximal end to the distal end of the catheter shaft for delivering hypothermically-cooled, physiologically-acceptable fluid from the fluid source to the patient's left or right coronary ostium. Optionally, two coronary perfusion catheters may be connected to the fluid source to allow simultaneous perfusion of both the right and left coronary arteries.

In one preferred embodiment, the coronary artery perfusion catheter includes one or more arch perfusion ports located on the exterior of the catheter shaft in the patient's aortic arch. Each arch perfusion port has a pressure-activated flow control valve for controlling fluid flow through the port(s). In addition, the selective coronary perfusion catheter may include an expandable flow control member located on the exterior of the catheter shaft in the patient's descending aorta. The expandable flow control member may be in the form of an inflatable balloon or a selectively-expandable external flow control valve.

The distal end of the coronary sinus perfusion catheter shaft is preferably curved to selectively engage the coronary sinus of the patient. A perfusion lumen extends through the catheter shaft from the perfusion fitting at the proximal end to the distal end of the catheter shaft for delivering hypothermically-cooled, physiologically-acceptable fluid from the fluid source to the patient's coronary sinus.

The coronary sinus perfusion catheter includes one or more perfusion ports located at or near the distal end of the catheter shaft. In addition, the coronary sinus perfusion catheter may include an expandable flow control member located on the exterior of the catheter shaft in the patient's coronary sinus. The expandable flow control member may be in the form of an inflatable balloon or a selectively-expandable external flow control valve. The sinus perfusion port(s) may have a pressure-activated flow control valve for controlling fluid flow through the port(s).

The fluid source may take one of several possible forms. In one preferred embodiment, the fluid source includes an arterial cannula for withdrawing autologously-oxygenated blood from the patient, a heat exchanger for hypothermically cooling the withdrawn blood and a blood pump for pumping the blood through the heat exchanger and the selective coronary perfusion catheter into the patient's coronary artery. In another preferred embodiment, the fluid source includes a venous cannula for withdrawing venous blood from the patient, a heat exchanger for hypothermically cooling the venous blood, a blood oxygenator for oxygenating the blood and a blood pump for pumping the blood through the heat exchanger, the blood oxygenator and the selective coronary perfusion catheter into the patient's coronary artery. Alternatively, the fluid source may include a supply of another physiologically-acceptable fluid, such as saline solution or an oxygenated blood substitute, and a fluid pump or pressure source for pumping the fluid through the selective coronary perfusion catheters into the patient's coronary artery. The fluid source may also include a heat exchanger for hypothermically cooling the fluid or the fluid may be precooled, for example by storing the fluid in a refrigerator.

The hypothermic fluid is alternatingly delivered to the coronary artery perfusion catheter and to the coronary sinus perfusion catheter. The configuration of the delivery system uses a single connector with a rotating body to alternate the flow. In the antegrade flow position, the hypothermic fluid is fed from the treatment station through a first valve passage and into the coronary artery perfusion catheter. Blood may also be withdrawn from the coronary sinus perfusion catheter. In recycling systems, the blood is then fed into the blood reservoir for treatment and re-entry into the body. In the retrograde flow position, the hypothermic fluid is fed from the treatment station through one of the valve passages and into the coronary sinus perfusion catheter. Simultaneously, blood may be withdrawn through the coronary arterial perfusion catheter. In recycling systems, the blood is then fed into the blood reservoir for treatment and re-entry into the body.

In other systems, the alternating delivery may use two separate systems: one for the coronary artery perfusion catheter and one for the coronary sinus perfusion catheter. In this version, one or more valves for each catheter would alternate the suction and perfusion cycles. The cycles between the arterial and sinus catheters would be timed to alternate the flow between antegrade and retrograde perfusion.

These and other features and advantages of the present invention will become apparent from the following detailed description of preferred embodiments which, taken in conjunction with the accompanying drawings, illustrate by way of example the present invention.

### BRIEF DESCRIPTION OF THE DRAWINGS

FIG. 1 is a schematic representation of the hypothermia system of the present invention;

FIG. 2 is a cutaway view of a patient's thoracic aorta showing a distal end of a coronary artery perfusion catheter positioned for delivering hypothermic fluid to the patient's myocardium;

FIG. 3 is a cutaway view of the patient's abdominal aorta showing a proximal end of the coronary artery perfusion catheter of FIG. 2;

FIG. 4 is a partial cutaway view of a patient's heart and coronary sinus showing a distal end of a coronary sinus perfusion catheter positioned for delivering hypothermic fluid to the patient's myocardium;

FIG. 5 is a cutaway view of the patient's femoral vein showing a proximal end of the coronary sinus perfusion catheter of FIG. 4;

FIG. 6 is a schematic diagram of a hypothermia system for delivering hypothermic fluid to the patient's myocardium using a supply of blood or another physiologically-acceptable fluid;

FIG. 7 is a schematic diagram of a hypothermia system for delivering hypothermic fluid to the patient's myocardium using hypothermically-cooled, autologously-oxygenated blood;

FIG. 8 is a schematic diagram of a hypothermia system for delivering hypothermic fluid to the patient's myocardium using hypothermically-cooled and oxygenated venous blood;

FIG. 9A shows a valve for directing the flow of hypothermic fluid to the coronary artery perfusion catheter;

FIG. 9B shows the valve of FIG. 9A rotated to direct the flow of hypothermic fluid to the coronary sinus perfusion catheter;

FIG. 9C shows an alternate version of the central valve body;

FIG. 10 shows a mechanically-actuated flow control valve for controlling fluid flow through a perfusion catheter shown in a closed position;

FIG. 11 shows the mechanically-actuated flow control valve of FIG. 10 in a open position; and

FIG. 12 shows an injection fitting with one-way valves for injection of a contrast medium and/or therapeutic agents through the lumen of one of perfusion catheters.

### DETAILED DESCRIPTION OF THE INVENTION

The present invention provides an apparatus and method for inducing hypothermia of the heart by hypothermic perfusion of the myocardium alternating between antegrade delivery via the coronary arteries and retrograde delivery via the coronary sinus. The apparatus and method provide rapid cooling of the affected myocardium to achieve optimal myocardial salvage in a patient experiencing acute myocardial infarction.

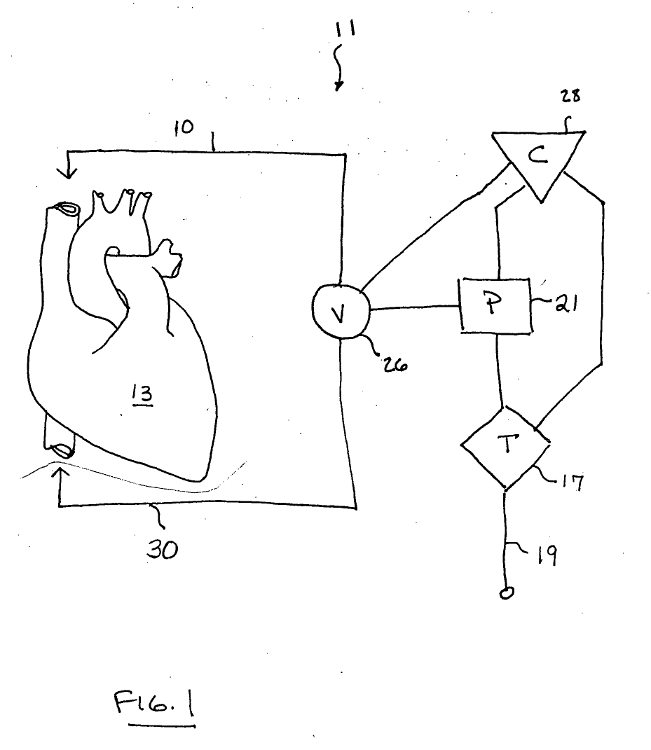

The apparatus generally takes the form of a system that includes at least one coronary artery perfusion catheter, a coronary sinus perfusion catheter and a fluid source for delivering a hypothermically-cooled physiologically-acceptable fluid via such catheters. FIG. 1 is a schematic representation of the hypothermia system 11 of the present invention. The heart 13 is catheterized with a coronary artery perfusion catheter 10 and a coronary sinus perfusion catheter 30. Blood drawn from the body, or optionally, fluid supplied from an outside source is routed to a treatment station 17 via conduit 19 where it is cooled and optionally oxygenated. A pump 21 forces the hypothermic fluid through valve 26, which alternates delivery either via catheter 10 or catheter 30. A programmable controller 28 receives input from any of a variety of sources including but not limited to from an operator, from temperature sensors that monitor the temperature of the heart, the temperature of the fluid prior to treatment, the temperature of the fluid after treatment, from oximeters measuring the oxygen content of the fluid prior to treatment, after treatment and after aspiration from the heart, from a sensor measuring the heart rate and from pressure and/or flow sensors measuring the flow rate of fluids through the various conduits. Such input is processed and used to control the operation of the valve, pump and treatment components of the system.

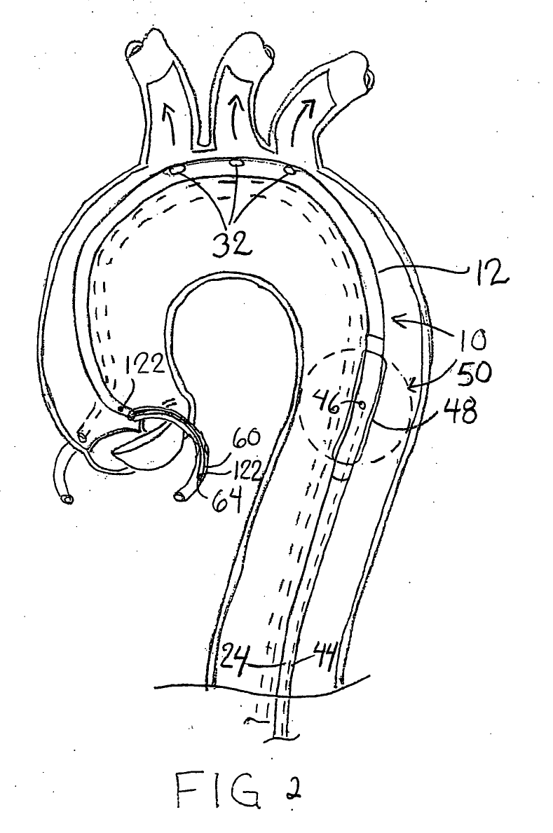

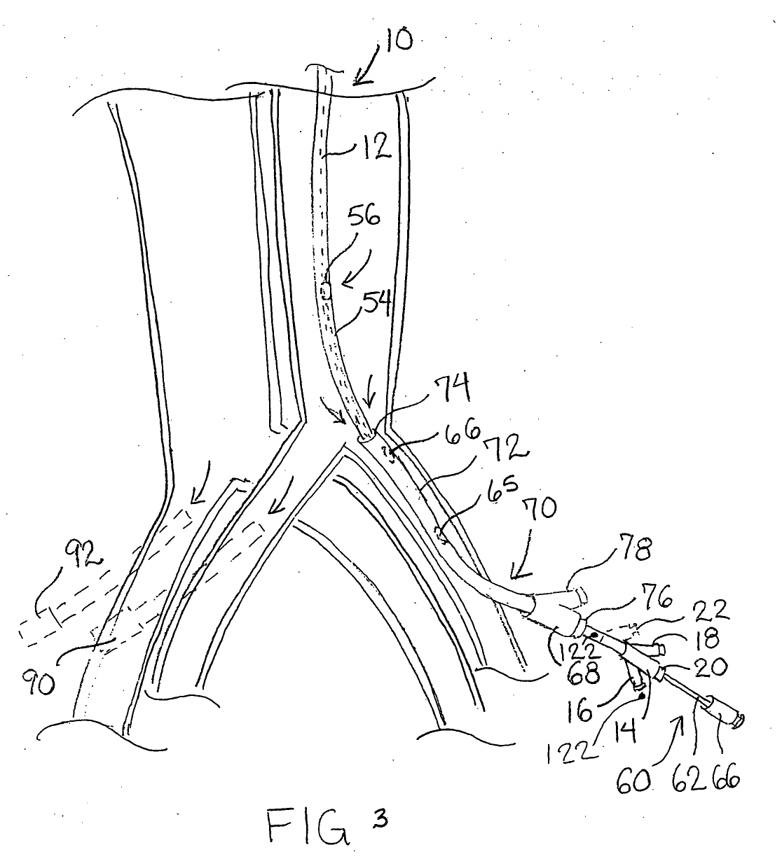

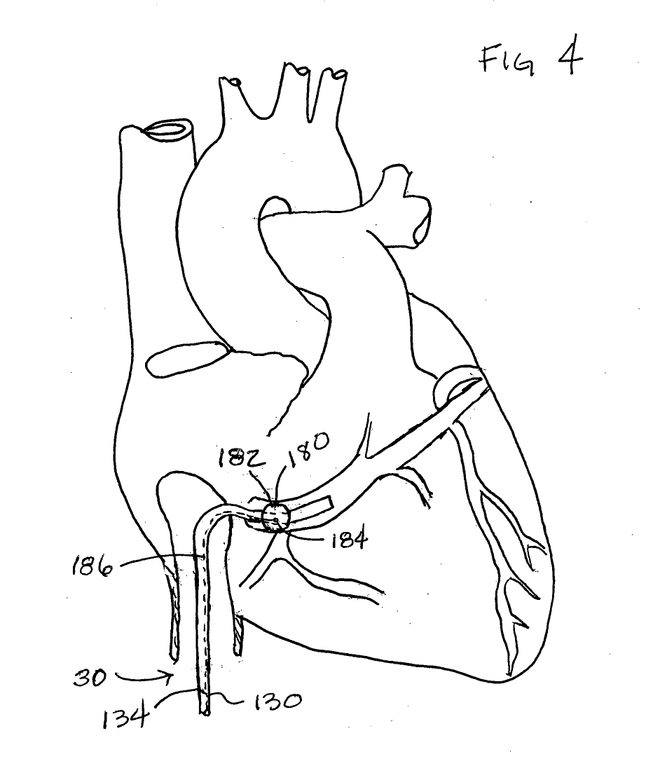

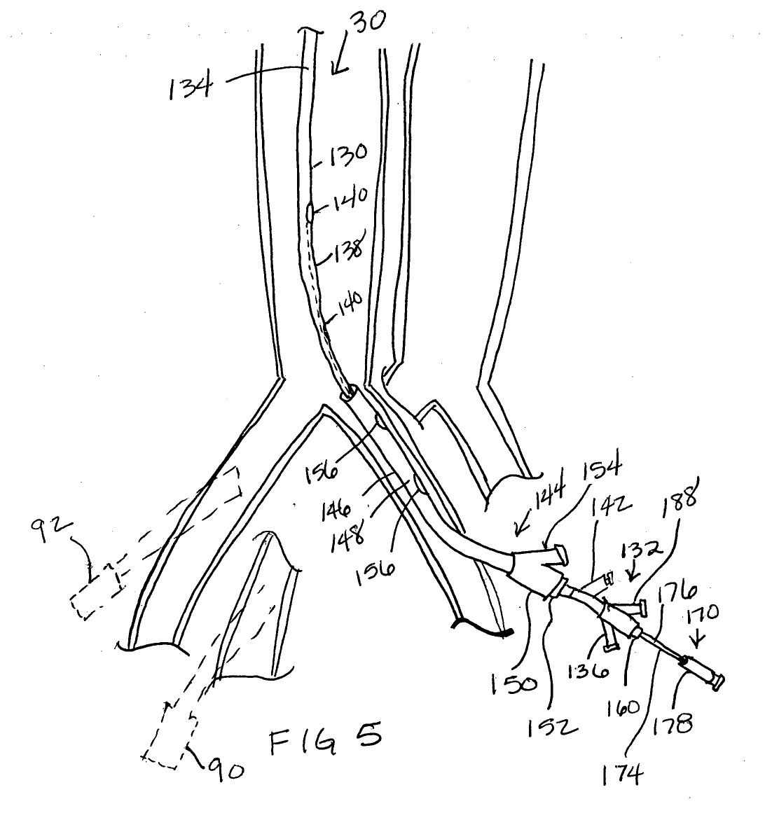

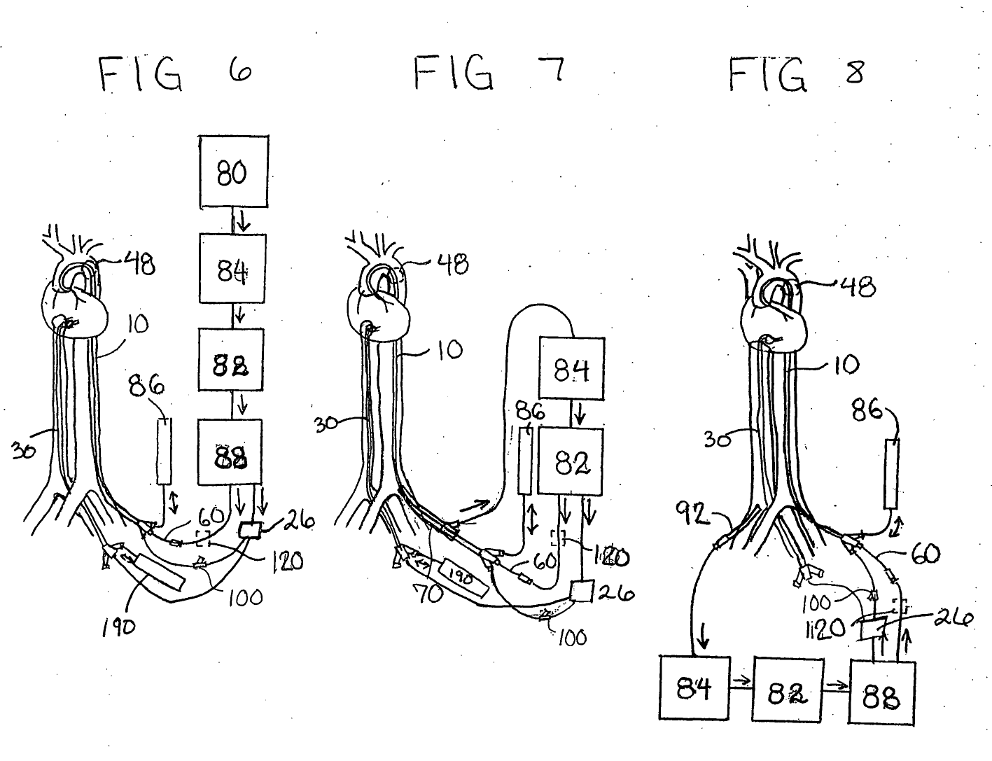

FIG. 2 is a cutaway view of a patient's thoracic aorta showing a distal end of a coronary artery perfusion catheter positioned for administering hypothermic fluid to the patient's myocardium. FIG. 3 is a cutaway view of the patient's abdominal aorta showing a proximal end of the coronary artery perfusion catheter 10 of FIG. 2. The coronary perfusion catheter 10 has an elongated catheter shaft 12 configured for transluminal introduction via an arterial insertion site, such as a femoral, subclavian or brachial artery. The catheter shaft 12 may be constructed of extruded polymeric tubing or, more preferably, of a fiber or wire braid-reinforced polymeric composite tubing. The catheter shaft 12 may have an outer diameter of approximately 1 to 3 mm and a length sufficient to extend from the arterial insertion site to the patient's aortic root. The length of the catheter shaft 12 may be from approximately 60 to 120 cm, depending on the arterial access site chosen. The distal end of the catheter shaft 12 is preferably curved to selectively engage either the right or the left coronary ostium or the catheter shaft 12 may be made with a multipurpose curve, which allows the operator to engage either coronary ostium. The proximal end of the catheter shaft 12 has a proximal fitting 14 configured for connecting to the fluid source. A perfusion lumen 24 extends through the catheter shaft 12 from a perfusion connector 16 on the proximal fitting 14 for delivering hypothermically-cooled, physiologically-acceptable fluid from the fluid source to the patient's left or right coronary artery. Optionally, two selective coronary perfusion catheters 10 may be connected to the fluid source to allow simultaneous perfusion of both the left and right coronary arteries.

In one preferred embodiment, the selective coronary perfusion catheter 10 includes one or more arch perfusion ports 32 located on the exterior of the catheter shaft 12 in the patient's ascending aorta and/or aortic arch. Preferably, the arch perfusion ports 32 are located near the superior aortic arch and directed upward toward the aortic arch vessels so that a majority of perfusate that exits the arch perfusion ports 32 enters the aortic arch vessels.

In addition, the coronary artery perfusion catheter 10 may include an expandable flow control member 50 located on the exterior of the catheter shaft 12 in the patient's descending aorta downstream of the aortic arch vessels. The expandable flow control member 50 may be in the form of an inflatable balloon 48, as shown, or in the form of a selectively-expandable external flow control valve. Selectively-expandable external flow control valves suitable for this application are described in U.S. Pat. No. 5,833,671, which is hereby incorporated by reference in its entirety. The interior of the inflatable balloon 48 is in fluid communication with a balloon inflation port 46 on the catheter shaft 12. A balloon inflation lumen 44 extends through the catheter shaft 12 from the balloon inflation port 46 to a balloon connector 18 on the proximal fitting 14.

The catheter shaft 12 is preferably made with a radiopaque construction, which facilitates viewing the catheter 10 by fluoroscopy. In addition, the catheter 10 may be constructed with one or more radiopaque and/or sonoreflective markers located along the catheter shaft 12 for visualizing the location of the distal tip of the catheter and the expandable flow control member 50 by fluoroscopy and/or ultrasonic imaging.

Optionally, the catheter 10 may be constructed with a blood withdrawal lumen 54 that extends through the catheter shaft 12 from one or more blood withdrawal ports 56 to a blood withdrawal connector 22 on the proximal fitting 14. Alternatively or in addition, the therapeutic hypothermia system may include an introducer sheath 70 for facilitating insertion of the selective coronary perfusion catheter 10 into the aorta from an arterial insertion site. Typically, the introducer sheath 70 will be constructed with a thin-walled tubular shaft 72 with a lumen 74 extending through it and a proximal fitting 68 with a hemostasis valve 76 or the like and a sidearm connector 78 for flushing the lumen 74 and/or for withdrawing arterial blood. Optionally, the introducer sheath 70 may have sideholes 65 in the tubular shaft 72 to facilitate blood entry into the lumen 74.

In addition, the proximal fitting 14 may be constructed with a hemostasis valve 20 or the like for introducing a guidewire and/or a subselective catheter 60 through the perfusion lumen 24 of the catheter 10. A subselective catheter 60 for use in the therapeutic hypothermia system may be configured as a flow guidewire, a subselective infusion catheter or an interventional catheter. For this application, the interior of the perfusion lumen 24 of the selective coronary perfusion catheter 10 will preferably have a lubricious or low friction surface to facilitate insertion of a catheter or guidewire through the catheter 10. When configured as a flow guidewire or subselective infusion catheter, the subselective catheter 60 will have an elongated shaft 62 with a lumen 64 extending through the shaft 62 from the proximal end to the distal end. Preferably, the exterior of the shaft 62 has a lubricious or low friction surface. A fitting 66 on the proximal end of the shaft 62 is configured for connecting the lumen 64 to a fluid source. The elongated shaft 62 is sized to fit through the perfusion lumen 24 of the selective coronary perfusion catheter 10 and may have an outer diameter of approximately 0.3 mm to 2 mm. The elongated shaft 62 has a length sufficient to extend through the perfusion lumen 24 of the selective coronary artery perfusion catheter 10 and advance distally beyond the distal end of the catheter shaft 12 into the patient's coronary artery. The flow guidewire or subselective catheter 60 can be used for administering subselective therapeutic hypothermia and/or for introducing an interventional catheter through the perfusion lumen 24 of the catheter 10. Subselective therapeutic hypothermia may also be administered through a lumen in the interventional catheter 60.

When the selective coronary perfusion catheter 10 is constructed with an inflatable balloon 48 as a flow control member, the system will preferably include an inflation/deflation device 86 for inflating and deflating the balloon 48. Optionally, the inflation/deflation device 86 may include means for synchronizing the inflation and deflation of the balloon 48 with the patient's heartbeat.

FIG. 4 is a partial cutaway view of a patient's heart and coronary sinus showing a distal end of a coronary sinus perfusion catheter 30 positioned for administering hypothermic fluid to the patient's myocardium. FIG. 5 is a cutaway view of the patient's femoral vein showing a proximal end of the coronary sinus perfusion catheter 30 of FIG. 4. The coronary sinus perfusion catheter 30 has an elongated catheter shaft 130 configured for transluminal introduction via a venous insertion site, such as a femoral or jugular vein. The catheter shaft 130 may be constructed of extruded polymeric tubing or, more preferably, of a fiber or wire braid-reinforced polymeric composite tubing. The catheter shaft 130 may have an outer diameter of approximately 1 to 5 mm and a length sufficient to extend from the femoral insertion site to the patient's coronary sinus. The length of the catheter shaft 130 may be from approximately 20 to 120 cm, depending on the venous access site chosen. The distal end of the catheter shaft 130 is preferably curved to engage coronary sinus. The proximal end of the catheter shaft 130 has a proximal fitting 132 configured for connecting to the fluid source. A perfusion lumen 134 extends through the catheter shaft 130 from a perfusion connector 136 on the proximal fitting 132 for delivering hypothermically-cooled, physiologically-acceptable fluid from the fluid source to the patient's coronary sinus.

The catheter shaft 130 is preferably made with a radiopaque construction, which facilitates viewing the catheter 30 by fluoroscopy. In addition, the catheter 30 may be constructed with one or more radiopaque and/or sonoreflective markers located along the catheter shaft 130 for visualizing the location of the distal tip of the catheter by fluoroscopy and/or ultrasonic imaging.

Optionally, the catheter 30 may be constructed with a blood withdrawal lumen 138 that extends through the catheter shaft 130 from one or more blood withdrawal ports 140 to a blood withdrawal connector 142 on the proximal fitting 132. Alternatively or in addition, the therapeutic hypothermia system may include an introducer sheath 144 for facilitating insertion of the selective coronary perfusion catheter 30 into the coronary sinus from a venous insertion site. Typically, the introducer sheath 144 will be constructed with a thin-walled tubular shaft 146 with a lumen 148 extending through it and a proximal fitting 150 with a hemostasis valve 152 or the like and a sidearm connector 154 for flushing the lumen 148 and/or for withdrawing arterial blood. Optionally, the introducer sheath 144 may have sideholes 156 in the tubular shaft 146 to facilitate blood entry into the lumen 148.

In addition, the proximal fitting 132 may be constructed with a hemostasis valve 160 or the like for introducing a guidewire and/or a subselective catheter 170 through the perfusion lumen 172 of the catheter 30. A subselective catheter 170 for use in the therapeutic hypothermia system may be configured as a flow guidewire, a subselective infusion catheter or an interventional catheter. For this application, the interior of the perfusion lumen 134 of the selective coronary sinus perfusion catheter 30 will preferably have a lubricious or low friction surface to facilitate insertion of a catheter or guidewire through the catheter 30. When configured as a flow guidewire or subselective infusion catheter, the subselective catheter 170 will have an elongated shaft 174 with a lumen 176 extending through the shaft 174 from the proximal end to the distal end. Preferably, the exterior of the shaft 174 has a lubricious or low friction surface. A fitting 178 on the proximal end of the shaft 174 is configured for connecting the lumen 176 to a fluid source. The elongated shaft 174 is sized to fit through the perfusion lumen 134 of the selective coronary perfusion catheter 30 and may have an outer diameter of approximately 0.3 mm to 2 mm. The elongated shaft 174 has a length sufficient to extend through the perfusion lumen 134 of the selective coronary sinus perfusion catheter 30 and advance distally beyond the distal end of the catheter shaft 130 into the patient's coronary sinus. The flow guidewire or subselective catheter 170 can be used for administering subselective therapeutic hypothermia and/or for introducing an interventional catheter through the perfusion lumen 134 of the catheter 30. Subselective therapeutic hypothermia may also be administered through a lumen in the interventional catheter 170.

In addition, the coronary sinus perfusion catheter 30 may include an expandable flow control member 180 located on the exterior of the catheter shaft 130 in the patient's coronary sinus. The expandable flow control member 180 may be in the form of an inflatable balloon 182, as shown, or in the form of a selectively-expandable external flow control valve. The interior of the inflatable balloon 182 is in fluid communication with a balloon inflation port 184 on the catheter shaft 130. A balloon inflation lumen 186 extends through the catheter shaft 130 from the balloon inflation port 184 to a balloon connector 188 on the proximal fitting 132. When the selective coronary perfusion catheter 30 is constructed with an inflatable balloon 182 as a flow control member, the system will preferably include an inflation/deflation device 190 for inflating and deflating the balloon 182. Optionally, the inflation/deflation device 190 may include means for synchronizing the inflation and deflation of the balloon 182 with the patient's heartbeat.

The fluid source for the hypothermia system may take one of several possible forms. FIG. 6 is a schematic diagram of a hypothermia system for delivering hypothermic fluid to the patient's myocardium that includes a fluid supply reservoir 80 containing a physiologically-acceptable fluid and a fluid pump 84 (or other pressure source, for example an intravenous reservoir pressurization cuff) for pumping the fluid through the selective coronary sinus catheter 30 and the selective coronary arterial perfusion catheter(s) 10 and/or the subselective catheter 60 into the patient's coronary artery or arteries. The fluid supply reservoir 80 may contain blood, saline solution, an oxygenated blood substitute or another physiologically-acceptable fluid.

Optionally, the therapeutic hypothermia system may include a heat exchanger 82 for hypothermically cooling the fluid from the fluid supply reservoir 80 before it enters the patient. Otherwise, the fluid may be precooled, for example by storing the fluid supply reservoir 80 in a refrigerator. This serves to simplify the therapeutic hypothermia system, which may save setup time in an emergency situation when the patient is in acute myocardial infarction. The therapeutic hypothermia system may also be prefilled with physiologically-acceptable fluid to facilitate setup in an emergency situation.

Optionally, the therapeutic hypothermia system may also include an oxygenator 88 for oxygenating the fluid from the fluid supply reservoir 80 before it enters the patient, such as when unoxygenated blood or an unoxygenated blood substitute are used. The use of a preoxygenated blood substitute, such as THEROX or PERFLUBRON, obviates the need for the oxygenator 88 and simplifies the system for faster setup in emergency situations.

FIG. 7 is a schematic diagram of a hypothermia system for delivering hypothermic fluid to the patient's myocardium using hypothermically-cooled autologously-oxygenated blood. Autologously-oxygenated arterial blood is withdrawn from the patient, pressurized by a blood pump 84, hypothermically cooled with a heat exchanger 82 and returned to the patient through the selective coronary sinus catheter 30 and the selective coronary arterial perfusion catheter(s) 10 and/or the subselective catheter 60 into the patient's coronary artery or arteries. The autologously-oxygenated arterial blood can be withdrawn from the patient through an introducer sheath 70 coaxial to the catheter 10, as shown in FIG. 6, and/or through a blood withdrawal lumen 54 within the catheter 10 or an arterial cannula 90, as shown in FIG. 3. An arterial cannula 90 can be placed in the contralateral or ipsilateral femoral artery and/or at another arterial access site. The use of autologously-oxygenated blood simplifies the system by eliminating the need for a blood oxygenator. In addition, the use of a coaxial introducer sheath 70 or a blood withdrawal lumen 54 within the catheter 10 simplifies the procedure and eliminates the need for making a second arterial puncture for placement of a separate arterial cannula 90. Simplifying the system and the procedure allows for faster setup and thus more rapid and effective therapy in emergency situations when the patient is in acute myocardial infarction.

FIG. 8 is a schematic diagram of a therapeutic hypothermia system for delivering selective hypothermia to the patient's myocardium using hypothermically-cooled and oxygenated venous blood. Venous blood is withdrawn from the patient through a venous cannula 92, pressurized by a blood pump 84, hypothermically cooled with a heat exchanger 82, oxygenated by a blood oxygenator 88 and returned to the patient through the selective coronary perfusion catheter(s) 10 and/or the subselective catheter 60 into the patient's coronary artery or arteries. The venous cannula 92 can be placed in the contralateral or ipsilateral femoral vein and/or at another venous access site.

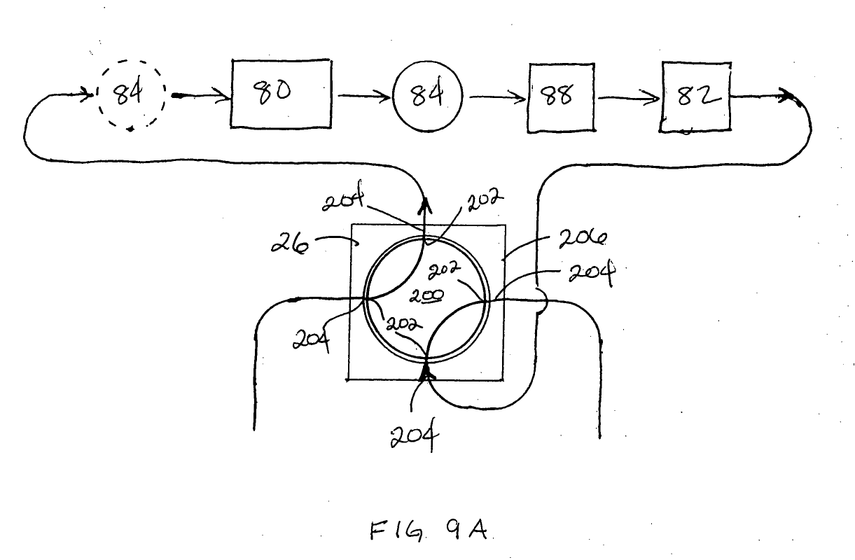

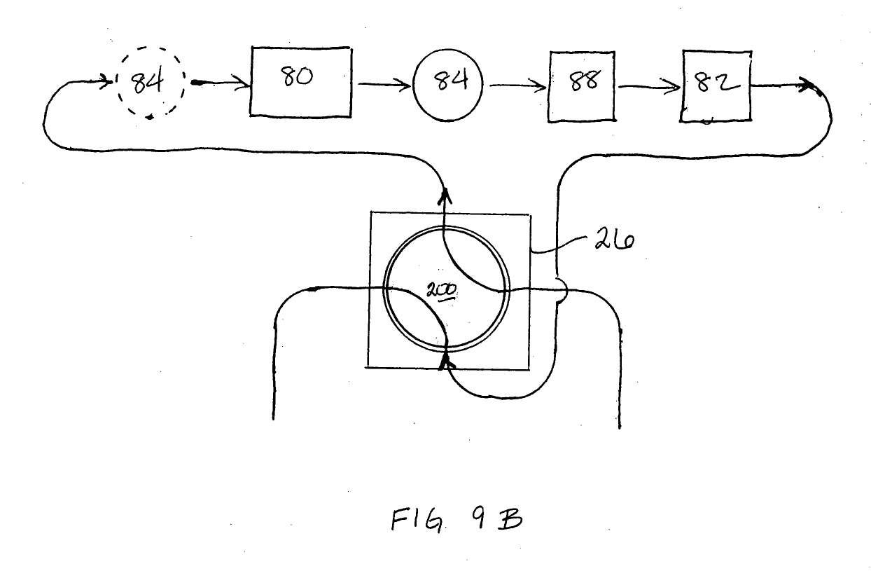

FIG. 9A and FIG. 9B show one version of the valve 26, which alternately feeds blood to the coronary artery perfusion catheter 10 and the coronary sinus perfusion catheter 30. In FIG. 9A, the valve is rotated to direct the flow of hypothermic fluid to the coronary artery perfusion catheter 10. When the central body 200 of the valve 26 is rotated a quarter turn, the valve is aligned to direct the flow of hypothermic fluid to the coronary sinus perfusion catheter 30. The central body 200 of the valve 26 has two curved passages. Each curved passage connects two of the adjacent openings 202 in the central body 200. The openings 202 are evenly spaced and configured to align with openings 204 in the valve housing 206. In this configuration of the valve 26, a quarter turn of the central valve body 200 switches from antegrade to retrograde flow and from retrograde flow to antegrade flow. For each 360 degree rotation of the valve, two antegrade and two retrograde cycles would be performed. However, it is not necessary to provide full rotation of the valve body. If preferred, the valve body 200 may rotate 90 degrees clockwise, then 90 degrees counter-clockwise, then back 90 degrees clockwise to provide the switches between antegrade and retrograde flow. If desired, the valve 26 may have sloped or graduated openings 210, as shown in FIG. 9C in the central body 200, thereby preventing pressure spikes during the rotation of the central body 200. In other embodiments, the valve 26 may take the form of a rotating piston, a reciprocating piston or other mechanism for switching the flow. Each opening 204 from the valve housing 206 includes a connector 212. In the embodiment shown, the connector 212 is a barbed fitting. However, the connector may also take the form of luer fittings, screw-type fittings, snap on connectors or other convenient fluid tight connections.

The cycle time of the valve would be selected for the patient and the particular needs of the situation. In one method, one antegrade and one retrograde cycle would take place for each heartbeat. The antegrade and retrograde cycle times could be made equal, thereby giving a cycle time of approximately 0.5 seconds for each flow direction. Alternately, the cycle times may be unequal, with either the antegrade or the retrograde taking up to 2 times or more of the reverse flow time. This would create a flow of 0.6 to 0.8 seconds in one direction and 0.4 to 0.2 in the reverse direction. The main cycle time and the amount of time spent in each flow direction may be set by the user. These times may also be altered for an initial period and changed one or more times during the procedure, based on the condition of the patient, feedback from temperature sensors or other automated or human input.

The valve 26 may also contain additional features to prevent undesirable effects such as pressure spikes. These additional features include a pressure overflow channel such as an opening in the central valve body 200, which would be located between the passage openings 202. The overflow channel would provide a buffer location for additional fluid to feed during rotation of the central valve body 200. A pressure overflow bypass may also be included. This would allow excess fluid fed into the valve 26 an additional exit passage. The overflow bypass exit opening would contain a pressure-sensitive valve to maintain a minimum pressure within the valve prior to allowing the release of fluid. If a preset pressure is reached, the pressure-sensitive valve would open to prevent over-pressurization of the fluid within the valve 26 and the catheters 10, 30.

The valve 26 and pump(s) 84 are configured to deliver from 50 to 300 mL/min in a pulsatile waveform. The amount delivered would vary depending on where the fluid is being fed. For example, if the left coronary artery is being perfused, a total of approximately 140 to 180 mL/min. could be used, thereby providing fluid for the left anterior descending and the circumflex. If the right coronary artery is being perfused, a total of approximately 80 to 100 mL/min could be used. Alternately, both the left and right coronary arteries may be perfused. For the coronary sinus, approximately 80 to 100 mL/min could be used. These amounts may be varied depending on the patient and the particular situation.

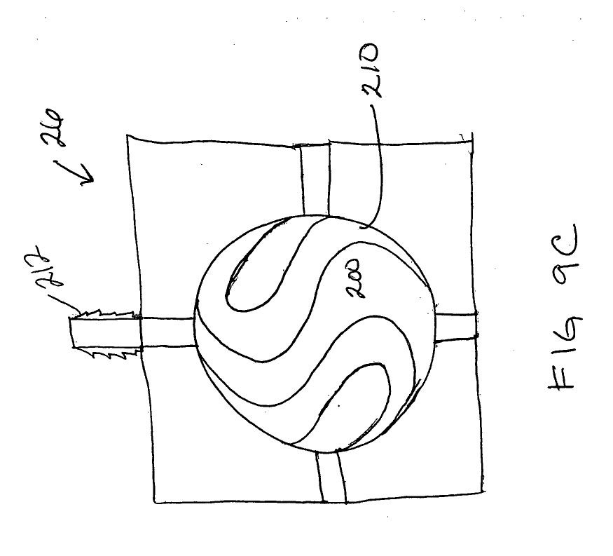

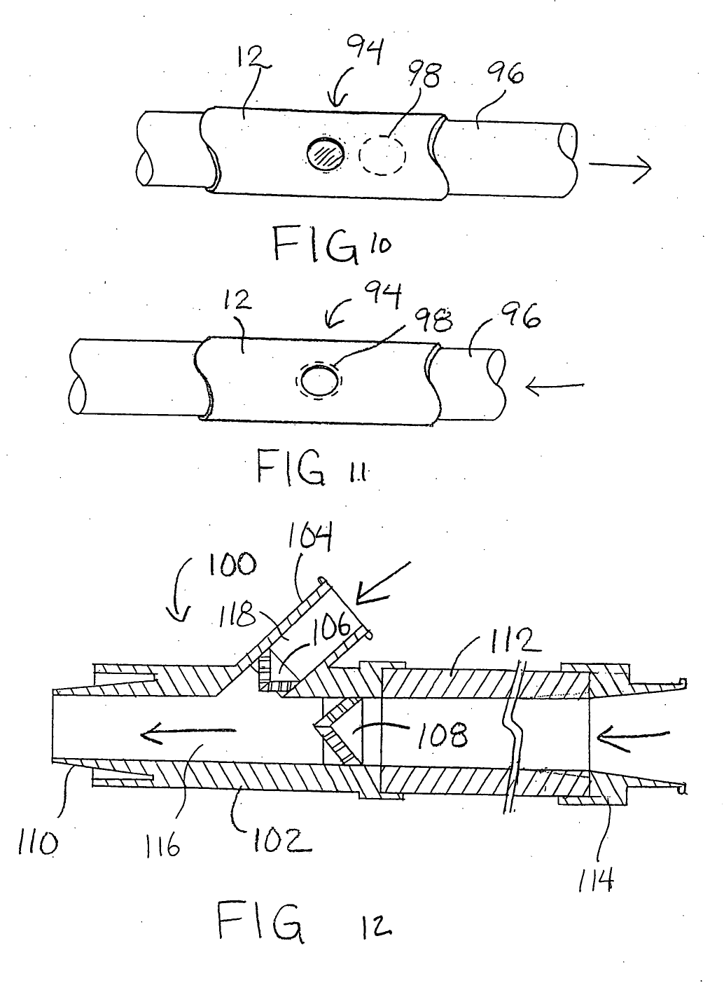

Preferably, each arch perfusion port 32 in has a mechanically or pressure activated flow control valve 94 for controlling fluid flow through the port(s) 32. FIGS. 10 and 11 are enlarged views of a portion of the catheter shaft 12 of the selective coronary arterial perfusion catheter 10 of FIG. 2 showing an arch perfusion port 32 with mechanically-actuated flow control valves 94 for controlling fluid flow through the exit port(s) 32. The mechanically-actuated flow control valve 94 includes a movable inner flap or sleeve 96 that covers the arch perfusion port 32. The distal end of the elastomeric sleeve 96 is affixed to the catheter shaft 12, while the proximal end of the elastomeric sleeve 96 is unattached. Preferably, the catheter 10 is constructed so that the elastomeric sleeve 96 is flush with the surface of the catheter shaft 12 when the pressure-activated flow control valve 94 is in a closed position. The inner sleeve 96 has aperture 98 through the wall thereof. FIG. 10 shows the mechanically-actuated flow control valve 94 in a closed position with the wall of the inner sleeve 96 blocking flow through the arch perfusion port 32. To open the mechanically-actuated flow control valve 94, the inner sleeve 96 is rotated and/or moved axially to align the aperture 98 in the inner sleeve 96 with the arch perfusion port 32 in the catheter shaft 12, as shown in FIG. 11. The elasticity of the elastomeric sleeve 96 may be selected so that the a pressure-activated version of the flow control valve 94 remains closed until the backpressure within the perfusion lumen 24 reaches a predetermined level, then the flow control valve 94 opens to allow excess perfusate to exit the arch perfusion ports 32. Alternatively, the elastomeric flap or sleeve 96 may be constructed with one or more pores that remain closed until the backpressure within the perfusion lumen 24 reaches a predetermined level, whereupon the pore(s) open to allow perfusate to exit the arch perfusion ports 32.

FIG. 12 shows an injection fitting 100 that may be utilized as part of the therapeutic hypothermia system on either the arterial side of the system, as shown, or the venous side of the system. The injection fitting 100 has a main body 102 with a main channel 116 running through it and a male luer lock, barb connector or the like 110 at the distal end of the main channel 116 and a female luer lock, barb connector or the like 114 at the proximal end of the main channel 116. A side branch 104 with a female luer lock connector or the like 110 has a side branch channel 118 that connects to the main channel 116. A first one-way check valve 108 is positioned in the main channel 116 proximal to the takeoff of the side branch 104. The first one-way check valve 108 is configured to allow fluid to flow in the distal direction through the main channel 116 and to prevent flow in the proximal direction in the main channel 116. A second one-way check valve 106 is positioned in the side branch channel 118. The second one-way check valve 106 is configured to allow fluid to flow in the distal direction through side branch channel 118 into the main channel 116 and to prevent flow in the proximal direction in the side branch channel 118. Optionally, the injection fitting 100 may include an elastomeric extension tube 112 connecting the main body 102 with the female luer lock 114 on the proximal end of the main channel 116. The elastomeric extension tube 112 can expand to serve as a fluid accumulator for perfusate in the main channel 116 when fluid is injected through the side branch 104 of the injection fitting 100.

Optionally, the injection fitting 100 may be connected in series with the perfusion connector 16 on the proximal fitting 14 of the selective coronary perfusion catheter 10, as shown in FIGS. 6, 7 and 8. The injection fitting 100 facilitates injection of a radiopaque contrast medium, therapeutic agents and/or other fluids through the perfusion lumen 24 of the selective coronary perfusion catheter 10 via the side branch 104 without interrupting the flow of perfusate through the main channel 116.

Higher injection pressures may be needed for perfusing fluids at adequate therapeutic flow rates through a small-diameter flow guidewire or subselective catheter 60 compared to the perfusion pressure needed for the selective coronary perfusion catheter 10. To compensate for this, an optional second blood flow pump 120 may be connected in series to boost perfusion pressure through the flow guidewire or subselective catheter 60, as shown in FIGS. 6, 7 and 8.

The method of the present invention can be used in an emergency situation for treating a patient in acute myocardial infarction with therapeutic hypothermia or it can be used electively to create a protective hypothermic environment for the patient's myocardium prior to, during or after performing a catheter-based intervention. To begin, one or more of the patient's coronary arteries is selectively catheterized using the selective coronary perfusion catheter 10 as described above. A diagnostic angiogram can be performed by injecting radiopaque dye through the selective coronary perfusion catheter 10 to determine the location and severity of any lesions in the coronary arteries. Meanwhile, the fluid source is set up according to one of the examples shown in FIGS. 6, 7 and 8. The proximal fitting 14 of the catheter 10 is connected to the fluid source and therapeutic infusion of hypothermically-cooled fluid is begun. For emergency situations, the system setup, catheterization and initiation of therapeutic hypothermia should be done as rapidly as possible in order to effectively salvage as much of the myocardium as possible.

In addition to the above, a flow guidewire or subselective catheter 60 may be introduced through the selective catheter 10 and advanced into the patient's coronary artery. Depending on the location and severity of the coronary lesion, the subselective catheter 60 may be advanced across the lesion for therapeutic infusion of hypothermically-cooled fluid to the threatened myocardium downstream of the lesion. Alternatively or in addition, a therapeutic catheter such as an angioplasty, atherectomy or stent delivery catheter may be introduced through the selective catheter 10 and advanced into the patient's coronary artery for treating one or more of the coronary lesions. The hypothermic environment created by the therapeutic hypothermia system protects the patient's myocardium, reducing the risk of any catheter-based intervention and reducing the likelihood of reperfusion injury to the myocardium downstream of the lesion.

Therapeutic agents, such as thrombolytic agents and pharmacological agents for reducing reperfusion injury, can be administered through the selective coronary perfusion catheter(s) 10 and/or the subselective catheter 60 into the patient's coronary artery or arteries. Optionally, a pharmacological agent effective to slow the patient's heartbeat without arresting the heart can be administered through the selective coronary perfusion catheter(s) 10 to reduce the metabolic demand of the myocardium, which may result in less ischemic damage and more effective myocardial salvage.

Preferably, the therapeutic hypothermia system cools the patient's myocardium to a temperature of approximately 28 to 36 C, more preferably to a temperature of approximately 32 to 35 C, to create a protective hypothermic environment without stopping the heart and without significant risk of nerve block or induced arrhythmias, which can be a consequence of more profound hypothermia. In one preferred method, an initial bolus of cold perfusate at a temperature of approximately 10 to 20 C may be infused to rapidly initiate therapeutic hypothermia, followed by steady infusion of perfusate at a temperature closer to the target temperature range of approximately 28 to 36 C or 32 to 35 C, depending on the clinical protocol that is selected. In another preferred method, a low flow rate of cold perfusate, for example saline solution, at a temperature of approximately 10 to 20 C may be added to the patient's native coronary blood flow to achieve an average temperature in the desired therapeutic temperature range.

Temperature feedback may be used to control the temperature and/or flow rate of the hypothermically-cooled fluid to achieve optimum therapeutic effect. Optionally, temperature sensors 122 may be incorporated into the therapeutic hypothermia system in the heat exchanger 82, in the proximal and/or distal end of the selective coronary artery perfusion catheter 10 and/or in the guidewire or subselective catheter 60, as shown in FIGS. 1 and 2 or on the proximal or distal end of the selective coronary sinus perfusion catheter 30. A feedback signal from the temperature sensor(s) 122 will be used to adjust the temperature and/or flow rate of the perfusate to achieve the desired tissue temperatures for effective therapeutic hypothermia.

When the backpressure within the perfusion lumen 24 of the selective coronary perfusion catheter 10 reaches a predetermined level, the flow control valve(s) 34 open to allow excess perfusate to exit the arch perfusion port(s) 32. In alternative embodiments, the mechanically-actuated flow control valve(s) 94 may be selectively opened to allow flow through the arch perfusion port(s) 32. The cold perfusate exiting the arch perfusion ports 32 mixes with the blood in the ascending aorta and aortic arch. Because the arch perfusion ports 32 are located near and directed upward toward the superior aortic arch, a majority of cold perfusate that exits the arch perfusion ports 32 enters the aortic arch vessels. This mechanism has two benefits. It prevents any potential damage from overperfusion of the coronary arteries and it provides a measure of hypothermic protection to the brain by way of the arch vessels.

If desired, the expandable flow control member 50 may be expanded to resist or to occlude blood flow in the patient's descending aorta downstream of the aortic arch vessels. This provides a greater proportion of the patient's cardiac output to the brain and the coronary arteries without significantly compromising the organ systems downstream of the aortic arch, which are much more resistant to ischemic damage.

Optionally, the expandable flow control member 50 may be synchronized with the patient's heartbeat. For example, when the expandable flow control member 50 is in the form of an inflatable balloon 48, the inflation/deflation device 86 may be constructed with means for synchronizing the inflation and deflation of the balloon 48 with the patient's heartbeat. The inflation/deflation device 86 may be synchronized using the patient's EKG signal or any other indicator of the cardiac cycle. The inflatable balloon 48 may be synchronized to inflate during diastole (counterpulsation), which will result in increased blood flow to the patient's coronary arteries. Alternatively, the inflatable balloon 48 may be synchronized to inflate during systole, which will result in increased blood flow to the patient's coronary arteries.

While the foregoing examples are provided as general guidelines for configuring the therapeutic hypothermia system, it will be apparent to one of ordinary skill in the art that many modifications, improvements and subcombinations of the various embodiments, adaptations and variations can be made to the invention without departing from the spirit and scope thereof. For example, some variation in the configuration and the order of the components in the fluid flow circuits may be acceptable. In addition, some or all of the components of the system may be combined to create a compact, integrated therapeutic hypothermia system.

1. A method for treating a patient experiencing acute myocardial infarction, comprising:
selectively catheterizing at least one of the patient's coronary arteries with at least one coronary artery perfusion catheter;
catheterizing the patient's coronary sinus with a coronary sinus perfusion catheter;
delivering hypothermic fluid alternately through the coronary artery perfusion catheter and the coronary sinus perfusion catheter to cool the patient's myocardium without stopping the patient's heart from beating.

2. The method of claim 1, wherein a first coronary artery perfusion catheter and a second coronary artery perfusion catheter are used to catheterize a first coronary artery and a second coronary artery.

3. The method of claim 1, wherein the hypothermic fluid comprises hypothermically cooled, oxygenated blood.

4. The method of claim 1, wherein the hypothermic fluid comprises hypothermically cooled, autologously oxygenated blood.

5. The method of claim 1, wherein the hypothermic fluid comprises hypothermically cooled saline solution.

6. The method of claim 1, wherein the hypothermic fluid comprises a hypothermically cooled, oxygenated physiologically acceptable solution.

7. The method of claim 1, wherein the hypothermic fluid comprises a hypothermically cooled, oxygenated blood substitute.

8. The method of claim 1, wherein the hypothermic fluid is delivered by a pump connected to the coronary artery perfusion catheter and the coronary sinus perfusion catheter.

9. The method of claim 8, wherein a flow switch alternately connects an outflow of the pump to the coronary artery perfusion catheter and the coronary sinus perfusion catheter.

10. The method of claim 1, wherein said hypothermic fluid is delivered at a constant temperature.

11. The method of claim 1, wherein said hypothermic fluid is delivered at a varying temperature.

12. The method of claim 11, wherein said temperature of said hypothermic fluid is gradually increased as the heart cools down.

13. The method of claim 2, further comprising:
perfusing the patient's coronary arteries with an initial bolus of cold saline solution through the first coronary artery perfusion catheter and the second coronary artery perfusion catheter and subsequently perfusing hypothermically-cooled, oxygenated blood alternately through the coronary artery perfusion catheters and the coronary sinus perfusion catheter to cool the patient's myocardium without stopping the patient's heart from beating.

14. The method of claim 8, wherein the hypothermic fluid is delivered through the coronary artery infusion catheter with a pulsatile waveform.

15. The method of claim 1, further comprising:
occluding the patient's coronary vein with the coronary sinus perfusion catheter.

16. The method of claim 1, further comprising:
occluding the patient's coronary vein with the coronary sinus perfusion catheter during perfusion of hypothermic fluid through the coronary sinus perfusion catheter.

17. The method of claim 1, further comprising:
aspirating fluid from the patient's coronary artery during perfusion of hypothermic fluid through the coronary sinus perfusion catheter.

18. The method of claim 17, further comprising:
aspirating fluid from the patient's coronary sinus during delivery of hypothermic fluid through the coronary artery perfusion catheter.

19. The method of claim 1, further comprising:
aspirating fluid from the patient's coronary sinus during delivery of hypothermic fluid through the coronary artery perfusion catheter.

20. A method for treating a patient experiencing acute myocardial infarction, comprising:
catheterizing a first coronary artery with a first coronary artery perfusion catheter;
catheterizing a second coronary artery with a second coronary artery perfusion catheter;
catheterizing the patient's coronary sinus with a coronary sinus perfusion catheter; and
alternatingly delivering hypothermic fluid through the coronary artery perfusion catheters and through the coronary sinus perfusion catheter so as to induce a state of protective hypothermia in the patient's myocardium without stopping the patient's heart from beating.

21. The method of claim 20, further comprising:
continuing to infuse hypothermic fluid alternately through the coronary artery perfusion catheters and the coronary sinus perfusion catheter to maintain a state of protective hypothermia in the patient's myocardium without stopping the patient's heart from beating.

22. A method for treating a patient experiencing acute myocardial infarction, comprising:
catheterizing a first coronary artery with a first coronary artery perfusion catheter;
catheterizing a second coronary artery with a second coronary artery perfusion catheter;
catheterizing the patient's coronary sinus with a coronary sinus perfusion catheter;
delivering an initial bolus of deeply hypothermic fluid through at least one of said coronary artery perfusion catheters;
delivering moderately hypothermic fluid through at least one of said coronary artery perfusion catheters; and
subsequently delivering hypothermic fluid through at least one said coronary artery perfusion catheters so as to induce a state of protective hypothermia in the patient's myocardium without stopping the patient's heart from beating.

23. The method of claim 22, further comprising:
continuing to deliver hypothermic fluid alternately through the first and second coronary artery perfusion catheters and the coronary sinus perfusion catheter to cool the patient's myocardium without stopping the patient's heart from beating.
